(19) United States
(12) Patent Application Publication
(10) Pub. No.: US 2026/0249472 A1
(43) Pub. Date: Aug. 27, 2026

(54) FACILITY AND METHOD FOR ALIGNING A MANIPULATOR WITH A GUIDE LIGHT BEAM

(71) Applicant: Siemens Healthineers AG, Forchheim (DE)

(72) Inventor: Oliver Hornung, Unterleinleiter (DE)

(21) Appl. No.: 19/548,120

(22) Filed: Feb. 24, 2026

(30) Foreign Application Priority Data
Feb. 25, 2025 (DE) 10 2025 107 073.3

(51) Int. Cl. B25J 9/16; B25J 9/00; B25J 19/02

(52) U.S. Cl. CPC B25J 9/1692; B25J 9/0009; B25J 9/161; B25J 9/1694; B25J 19/022

(57) ABSTRACT

Systems and methods for the alignment of medical instruments and manipulators for medical instruments with a guide light beam, for example a laser beam. An alignment facility is used for aligning a manipulator with a guide light beam. The system includes a first projection surface configured to produce a first projection of a guide light beam incident on the first projection surface and an optical detection facility configured to detect a first projection produced on the first projection surface. The first projection surface is configured to be arranged on the manipulator in a fixed relative spatial position with respect to the manipulator in relation to at least two spatial axes. The optical detection facility is configured to be arranged on the manipulator in a fixed relative spatial position with respect to the first projection surface.

### CROSS REFERENCE TO RELATED APPLICATIONS

This application claims the benefit of DE 10 2025 107 073.3 filed on Feb. 25, 2025, which is hereby incorporated by reference in its entirety.

### FIELD

Embodiments relate to the alignment of medical instruments and manipulators for medical instruments with a guide light beam, for example a laser beam.

### BACKGROUND

One problem to be solved relates to the precision and accuracy of the alignment and placement of medical instruments by a manipulator.

It is known to use laser systems to correctly align medical instruments for diagnosis or treatment, for example needles or sleeves, by a manipulator. Herein, laser systems emit a laser beam or laser fan to guide a user when aligning an instrument. Such laser systems are, for example, used to align ablation needles and biopsy needles. This process is also known as needle guidance.

Known laser systems do not provide quantitative measurement results for the alignment of needles or sleeves. Therefore, they cannot guarantee reproducible, precise alignment results with optimum accuracy. Known laser systems are used for qualitative guidance. For example, for needle guidance, the distal needle tip is first aligned with the projection of a laser cross on the skin. The laser cross is projected onto the skin in exactly the direction or orientation in which the needle is to be inserted. Once the needle tip is aligned with the projection of the laser cross, the proximal section of the needle is moved around this pivot point in a swiveling motion. Herein, the proximal section of the needle is aligned with the laser cross. As a result, the entire length of the needle is then aligned with the laser cross and thus assumes the same orientation as the line of intersection of the two laser fans forming the laser cross.

Once the needle is aligned, it may be inserted into the body. Experience has shown that displacements of the entry point, short distances between the entry point and the proximal end of the needle or particularly deep insertion into the body may lead to inferior results. Therefore, frequent checks using X-ray images are necessary to monitor the progress of the procedure and the alignment of the needle. For this purpose, for example in the case of 2D imaging systems, X-ray images often have to be taken from different projection angles in order to obtain a spatial impression. This leads to a cumbersome and time-consuming workflow and increases the X-ray dose.

### BRIEF SUMMARY AND DESCRIPTION

The scope of the present disclosure is defined solely by the claims and is not affected to any degree by the statements within this summary. The present embodiments may obviate one or more of the drawbacks or limitations in the related art. Independent of the grammatical term usage, individuals with male, female or other gender identities are included within the term.

Embodiments provide a more reliable and precise method for aligning a manipulator for medical instruments. The technical problem is to improve the accuracy and reproducibility of the alignment of the manipulator by incorporating a quantitative measurement approach that overcomes the limitations of qualitative guidance provided by existing laser projection systems.

According to an embodiment, a projection surface for the guide light beam of a guide light system and an assigned optical detection facility arranged on the manipulator are used. This configuration ensures that accurate alignment of the manipulator may be achieved, regardless of the respective pose of the manipulator or the gravitational forces acting on the manipulator. Embodiments thus improve the precision of the alignment of the instrument and provides a quantitative measurement result for the alignment.

Embodiments further relate to an alignment facility for aligning a manipulator with a guide light beam. Embodiments provide precise and repeatable alignment of the manipulator by using a first projection surface and an optical detection facility. The fact that the manipulator may be aligned repeatedly and precisely means that an instrument held and guided by the manipulator may also be aligned repeatedly and precisely. Herein, the term “manipulator” may be understood as meaning an apparatus that allows an instrument held by the manipulator to be positioned and aligned by moving the manipulator. A manipulator may, for example, be a suitable kinematic device, a tripod or a robot.

One problem to be solved is the need for accurate and stable alignment of medical instruments or other apparatuses arranged on a manipulator in relation to a guide light beam. Traditional methods of alignment may be susceptible to inaccuracies caused by movements or vibrations of the manipulator. These inaccuracies may significantly impair efficiency and precision.

The alignment facility includes a first projection surface on which a projection of a guide light beam incident thereon is produced. An optical detection facility detects this projection and provides for the position of the projection within the first projection surface to be determined. The first projection surface is attached to the manipulator in a fixed manner in order to provide a stable spatial reference.

Advantageously, the use of a projection surface and an optical detection facility allows high accuracy to be achieved in determining the position of the guide light beam. The precise optical determination of the position of the guide light beam or its projection reduces the probability of alignment errors.

The optical detection facility may be a camera aligned with the projection surface. For example, it may be an optical camera such as those used for visual recording and monitoring of the alignment of manipulators and medical instruments. It may record images or videos and be used in conjunction with other systems such as lasers and projection surfaces. It may record the projection of a laser or light beam, for example a guide light beam, onto a projection surface. Alternatively, the optical detection facility may also be a light-sensitive layer that is sensitive to the wavelength of the guide light beam. Such a sensitive layer may be arranged on the respective projection surface or integrated with the respective projection surface. A sensitive layer of this kind may be considered to be an image sensor, so to speak. Instead of an optical detection facility, it may alternatively also be referred to as an image recording system or an optical camera system or an image sensor. An example of an optical detection facility is a high-resolution camera that is used to monitor the alignment and position of a needle during a medical procedure.

According to an aspect, the alignment facility is arranged detachably on the manipulator. This makes it possible to detach and remove it after the alignment of the manipulator before the manipulator is used for its actual intended purpose.

A fixed arrangement of the alignment facility on the manipulator provides stable and repeatable precise alignment of the manipulator. However, depending on the intended use of the manipulator, the alignment facility may be disruptive or obstructive during subsequent use of the manipulator. For example, the manipulator may be used in a sterile medical environment requiring the alignment facility also to be kept sterile. For example, the manipulator may be used in a spatially confined area and the alignment facility might obstruct valuable space and represent an obstacle.

This also provides for the alignment facility to be detached after the alignment of a manipulator and then used to align further manipulators. This allows the same alignment facility to be used to align not only a single manipulator but also a plurality of further manipulators.

According to an aspect, the alignment facility is configured to be arranged on the manipulator in a fixed relative spatial position with respect to the manipulator.

The fixed arrangement of the projection surfaces on the manipulator ensures stable and repeatable alignment. This stability contributes significantly to the reliability and consistency of the alignment processes. It also facilitates ease of use, since there is no additional need for the user to adjust the alignment facility on the manipulator. This saves time and reduces the effort required by the user.

In the aspects described below, in which projections of the guide light beam are used in different positions of the projection surface, a different position of the projection surface is achieved in each case with a spatially fixed arrangement of the alignment facility by moving the manipulator. This also advantageously incorporates the movement of the manipulator into the alignment method. If the movement of the manipulator exhibits inaccuracies or tolerances, these may be reflected in inaccuracies or tolerances in the positioning of the projection surface and may therefore be taken into account in further alignment.

According to an aspect the alignment facility is configured to be arranged on the manipulator in a linearly displaceable relative spatial position with respect to the manipulator.

Linear displaceability increases the versatility of the alignment facility. At the same time, restriction to linear movement ensures increased precision of movement.

In the aspects described below, in which projections of the guide light beam are used in different positions of the projection surface, a different position of the projection surface is easily achieved by moving the alignment facility. This advantageously prevents possible inaccuracies or tolerances in the movements of the manipulator. These inaccuracies or tolerances may therefore also not be reflected in the alignment method. This allows the precision of the alignment to be increased.

According to an aspect, the first projection surface is transparent. “Transparent” means that, on the one hand, a projection of the guide light beam may be produced on the projection surface, but, on the other hand, a portion of the light that is not reflected by the projection may pass through the projection surface. More precisely, “transparent” means partially transparent or semi-transparent. This allows the portion of the guide light beam passing through the projection surface to be used additionally after passing through. In aspects described below, the portion of the guide light beam passing through is used to produce a second projection on a second projection surface.

According to an aspect the alignment facility additionally includes a second projection surface. Herein, the first and a second projection surface are arranged one above the other at a fixed distance from one another. Herein, “one above the other” means that the planes in which the projection surfaces are located are arranged one above the other. In the simplest case “one above the other” means that the two projection surfaces are separated from one another by a vertical distance. Due to the arrangement one above the other, a guide light beam may first strike the first projection surface, pass through it, and then strike the second projection surface. Both projection surfaces are configured to produce a projection of the guide light beam. The optical detection facility is configured to detect both projections produced on the projection surfaces.

The use of two projection surfaces in combination with the optical detection facility provides exceptionally high accuracy in determining the position and angle of the guide light beam. This precision significantly improves the control and alignment of manipulators. The arrangement of two projection surfaces on the manipulator ensures stable and repeatable alignment without the need to move the alignment facility or the manipulator between the detection of the first and the second projection of the guide light beam through the projection surfaces. The use of a second projection surface allows even more precise determination of the respective position of the projection of the guide light beam within the projection surfaces. Based on the relative spatial position of the two projections to one another, the angle at which the guide light beam strikes the projection surfaces may be determined even more precisely.

The fixed arrangement of both projection surfaces and the optical detection facility on the manipulator eliminates the need for additional adjustments of the alignment facility or multiple positions of the alignment facility by the user. This saves time and reduces the effort required by the user.

According to an aspect, the distance between the two projection surfaces is 10 to 50 mm. This provides a small and compact design of the alignment facility in the order of one or a few centimeters. In addition, the minimum distance provides for a sufficiently accurate determination of the angle of incidence of the guide light beam based on the relative spatial positions of the projections of the guide light beam on the respective projection surface.

According to an aspect, the optical detection facility is configured as a layer that is sensitive to the wavelength of the guide light beam, and the respective projection surface includes this layer or is connected thereto. The use of a light-sensitive layer simplifies the design since a camera is no longer necessary. In addition, the effort of aligning a camera on the respective projection surface is eliminated. Furthermore, the effort required for optically calibrating camera imaging ratios is eliminated.

According to an aspect, the alignment facility includes a computing facility. The computing facility is configured to receive and process projection information from the optical detection facility in order to ascertain precise alignment information. As described below, the computing facility may, for example, ascertain the positions of multiple projections of the guide light beam within the projection surfaces in different projection surface positions. The computing facility may then use these positions to ascertain the angle at which the projection surfaces are aligned with the guide light beam. The computing facility supplements and completes the alignment facility, since it ascertains the ultimately relevant alignment information in the form of the alignment angle.

According to an aspect, the computing facility is configured to receive and process projection information from the optical detection facility in order to ascertain precise alignment information. The computing facility may, for example, ascertain the positions of multiple projections of the guide light beam within the projection surfaces in different projection surface positions. Based on these positions, the computing facility may then ascertain the angle at which the projection surfaces are aligned with the guide light beam. The computing facility supplements and completes the alignment facility, since it ascertains the ultimately relevant alignment information in the form of the alignment angle. This alignment information enables the angular alignment of the manipulator with the guide light beam to be determined accurately. The alignment angle may be used to derive precisely the angle by which the alignment of the manipulator has to be changed in order to obtain the desired or necessary alignment in relation to the guide light beam.

According to an aspect, the computing facility is configured to initiate a linear spatial displacement of the alignment facility. For example, after receiving a first item of information from the optical detection facility, the computing facility may therefore initiate displacement in order to then receive a second item of information from the detection facility. The displacement may be used to receive information from the detection facility in relation to the respective projection of the guide light beam in two projection surface positions located one above the other.

According to an aspect, the alignment facility is arranged in an alignment apparatus. In the form of the alignment apparatus, an apparatus component is created which contains the most important or even all components of the alignment facility. The alignment apparatus may be regarded as a standalone apparatus module which is arranged on a manipulator. The alignment apparatus may also be arranged detachably on a manipulator and, after use on a manipulator, detached and then re-attached to the same manipulator or to another manipulator or instrument for further use.

Embodiments also relates to a method for aligning a manipulator with a guide light beam using an alignment facility as described above. According to the method, in a first step, a first projection of a guide light beam incident on the first projection surface produced in a first projection surface position is detected by the optical detection facility. In a further step, a second projection of the guide light beam produced on the first or second projection surface in a second projection surface position is detected by the optical detection facility, wherein the first and the second projection surface position are located one above the other at a distance from one another. In a further step, first and second position information describing the respective position of the first and second projection within the respective projection surface is ascertained. In a further step, the computing facility uses the first and second position information to ascertain an angle between the guide light beam and the projection surface(s). The angle is then provided.

The method provides precise and repeatable alignment of the manipulator based on a first projection surface and the detection of a projection of a guide light beam on the projection surface. By providing for the manipulator to be aligned repeatably and precisely, an instrument held and guided by the manipulator may also be aligned repeatedly and precisely. By aligning the manipulator, an instrument or other apparatus held or guided thereby may thus be indirectly aligned in relation to a guide light beam.

The method includes the optical detection of multiple projections of the guide light beam produced on a projection surface in different projection surface positions. The optical detection may be used to ascertain the position of the respective projection within the respective projection surface. Advantageously, high accuracy may be achieved by using a projection surface and optically detecting the positions of the projections produced by the guide light beam.

The distance between the projection surface positions and the respective positions within the projection surfaces result in two spatial points that represent a vector with the direction of the guide light beam. Therefore, this means that the direction of the guide light beam in relation to the projection surface, and thus in relation to the alignment facility, may be ascertained optically with high accuracy. Based on the actual alignment ascertained on this way and with knowledge of the desired alignment in relation to the guide light beam, it is possible to deduce the angle by which the manipulator or the alignment facility has to be rotated in order to achieve the desired alignment.

According to an aspect, the method includes linearly moving the alignment facility with respect to the manipulator after the detection of the first projection and before the detection of the second projection of the guide light beam in such a way that the first projection surface is moved from one projection surface position to the other projection surface position in each case. In this way, a changed position of the projection surface is achieved. By displacing the alignment facility itself and not the manipulator, it is advantageously possible to prevent inaccuracies or tolerances in the movements of the manipulator. These inaccuracies or tolerances are therefore also not reflected in the alignment method. This provides for the precision of the alignment to be increased.

According to an aspect, the method includes linearly moving the manipulator after the detection of the first projection and before the detection of the second projection of the guide light beam in such a way that the first projection surface is moved from one projection surface position to the other projection surface position in each case. In this way, a changed position of the projection surface is used and a changed position of the projection surface is achieved. Moving the manipulator and not the alignment facility advantageously also includes the movements of the manipulator in the alignment method. If movements of the manipulator exhibit inaccuracies or tolerances, these may be reflected in inaccuracies or tolerances in the positioning of the projection surface and may therefore be taken into account in further alignment.

### BRIEF DESCRIPTION OF THE FIGURES

FIG. 1 depicts an alignment facility according to an embodiment.

FIG. 2 depicts an alignment facility according to an embodiment.

FIG. 3 depicts an alignment facility with a changed projection surface position according to an embodiment.

FIG. 4 depicts an alignment apparatus with two projection surfaces according to an embodiment.

FIG. 5 depicts an alignment apparatus with a laser-sensitive layer according to an embodiment.

FIG. 6 depicts a method according to an embodiment.

FIG. 7 depicts a method according to an embodiment.

### DETAILED DESCRIPTION

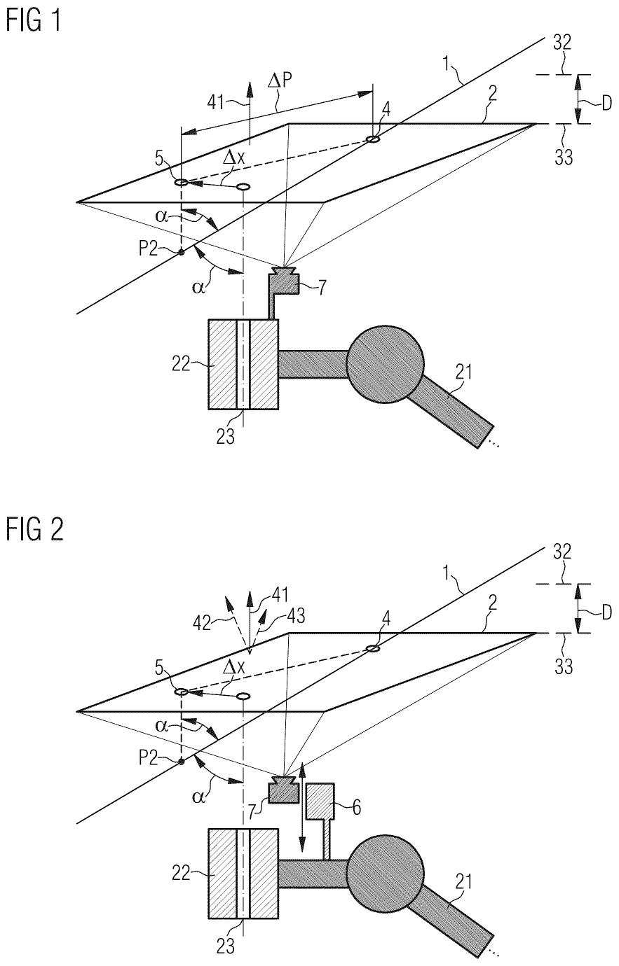

FIG. 1 depicts a schematic illustration of an alignment facility according to an embodiment. The alignment facility is arranged on a robotic arm (21) that is a component of a manipulator. The alignment facility may be arranged permanently or detachably on the robotic arm (21). The alignment facility may also be arranged indirectly on the robotic arm (21) by being attached to the medical instrument (22) held by the robotic arm (21); this instrument is configured as a needle guide sleeve. The robotic arm (21) aligns the needle guide sleeve (22) precisely with respect to a guide light beam (1). The alignment facility is arranged in a spatially stable manner in relation to the needle guide sleeve and thus indirectly to the robotic arm (21).

The guide light beam (1) may, for example, be a laser beam that is used to guide and align medical instruments. The guide light beam (1) may be used for projection onto one or more projection surfaces (2) in order to ensure the precise placement of instruments (22) such as needles or sleeves. Alternatively, guide light beam (1) may be referred to as a guidance light beam or alignment light beam. One example of a guide light beam (1) is a laser beam, such as those used for needle guidance, for example. In the following, the terms guide light beam and guide laser beam are used interchangeably.

The alignment facility includes a first projection surface (2). A projection surface may for example be a surface onto which a light beam, for example a laser beam, may be projected. The projection of a guide light beam (1) using a projection surface may facilitate the alignment of medical instruments (22). The projection surface may be flat or curved and made of various materials, such as glass or plastic. It may be transparent, semi-transparent, partially transparent or non-transparent. Alternatively, it may also be referred to as a projection plane or projection screen.

An optical detection facility configured as a camera (7) is aligned with the first projection surface (2). An optical detection facility may for example be an optical camera that is used for visual recording and monitoring of the alignment of manipulators and medical instruments. An optical detection facility may record images or videos and may be used in conjunction with other systems, such as lasers and projection surfaces. It may record the projection of a laser or light beam, for example a guide light beam (1), onto a projection surface (2). Recording the projection of a guide light beam (1) on a projection surface (2) thus also provides for the position (4) of this projection on the projection surface (2) to be determined. Instead of an optical detection facility, it is alternatively also possible to refer to an image recording system or an optical camera system or an image sensor. One example of an optical detection facility is a high-resolution camera that is used to monitor the alignment and position of a needle during a medical procedure.

The camera (7) is aligned with the first projection surface (2) in such a way that it may be used to detect projections produced on the first projection surface (2). As a result, the position (4) of a projection within the projection surface (2) may also be ascertained using the camera recording.

For example with the projection of a guide light beam, the projection of a guide light beam (1) may be understood as the mapping of the guide light beam (1) on a projection surface. Depending upon the shape of the guide light beam (1), the projection may take the form of a dot, cross, fan or another pattern. The projection of a guide light beam (1) may serve as visual assistance in the alignment of, for example, manipulators for medical instruments, for example needle guide manipulators or surgical manipulators, or in the alignment of the medical instruments themselves. Alternatively, it is also possible to refer to laser projection or laser beam projection. One example of guide light beam projection is the projection of a laser cross onto the skin for needle guidance.

The relative spatial position of the camera (7) in relation to the first projection surface (2) is specified by the structural arrangement of the camera (7) and the first projection surface (2) in relation to one another in the alignment facility. The relative position of the first projection surface (2) in relation to the camera (7) may be specified as unchangeable depending on the intended mode of operation of the alignment facility. However, it may also be changeable by changing the position of the first projection surface (2) in relation to the camera (7). In this case, the optical mapping ratios of the camera (7) may change, and this may have to be taken into account when evaluating the position (4) based on the projection of the guide light beam (1). For example, depending on the mode of operation of the alignment facility, the distance between the first projection surface (2) and the camera may be changeable. The structural components necessary for the arrangement of the camera (7) and the first projection surface (2) are not shown in the schematic illustration.

A guide light beam (2) in the form of a guide laser beam strikes the first projection surface (2) at a specific position (4) within the first projection surface (2). The guide laser beam (1) produces a first laser beam projection in this position (4). The first laser beam projection becomes visible because the light of the guide laser beam is at least partially refracted or reflected by the first projection surface (2). In contrast, the guide laser beam (1) itself is generally not visible, at least not when it passes through clear air. The first projection surface (2) is transparent or semi-transparent or partially transparent, such that the first laser beam projection is visible on both sides of the first projection surface (2). Here and in the following, the terms transparent, semi-transparent and partially transparent are used interchangeably and refer to the property of the first projection surface (2) to allow light or laser light to pass through at least partially and to partially refract or reflect it.

The first laser beam projection may be detected by the camera (7). This means that the position (4) of the first laser beam projection within the first projection surface (2) may also be ascertained using the camera (7). This means a single position of the guide laser beam (1) is known in relation to the first projection surface (2) and thus to the alignment facility and thus to the robotic arm (21).

However, to be able to ascertain the angle α in which the guide laser beam (1) strikes the first projection surface (2), at least one further spatial position of the guide laser beam (1) must be ascertained. For this purpose, the alignment facility is moved linearly, i.e., along a single spatial axis, by the robotic arm (21). The movement is orientated in such a way that the first projection surface (2) assumes another projection surface position (32, 33) above or below it. The first projection surface (2) is therefore not moved within its plane but out of this plane. For example, the movement may be a vertical movement perpendicular to the initial plane. As the result of the movement, the first projection surface (2) assumes a changed projection surface position (32, 33) that is at a distance D from the previous projection surface position (32, 33) the distance D. In the schematic illustration, the first projection surface (2) is shown in only one position (33).

The change in position (32, 33) of the first projection surface (2) causes a non-perpendicularly striking guide laser beam (1) to strike the first projection surface (2) at a different position (5). A guide laser beam (1) that strikes perpendicularly may not change its position within the first projection surface (2) if the first projection surface (2) were displaced perpendicularly. A second laser beam projection is now produced at the different position (5). The second laser beam projection is also detected by the camera (7) so that its position (5) within the first projection surface (2) may be ascertained. Then, the respective position (4, 5) of the first and second laser beam projection and of the distance D may be used to calculate the angle α at which the guide laser beam (1) strikes the first projection surface (2) and to determine its direction. The angle α may be calculated according to the law of sines (tan α=ΔP/D).

In addition to calculating the angle α and its spatial alignment, the ascertained positions (4, 5) of the first and second laser beam projection also enable the calculation of a correction distance Δx. While the angle α indicates the angle by which the robotic arm (21) must be tilted in order to achieve the desired alignment, the correction distance Δx indicates the distance by which the robotic arm (21) must be displaced.

In the example illustrated, the alignment to be achieved is such that the needle guidance (23) is aligned with the guide laser beam (1). If the robotic arm (21) is merely tilted, although the needle guidance (23) may be aligned parallel to the orientation of the guide laser beam (1), it may possibly be displaced laterally relative thereto. The correction distance Δx indicates the lateral displacement required in relation to the guide laser beam (1) in order to not only align the needle guidance (23) parallel to the guide laser beam (1), but also to cause it to overlap therewith. In other words, moving the robotic arm (21) laterally by the correction distance Δx causes the needle guidance (23), which is already aligned parallel, to be aligned coaxially in such a way that the guide laser beam (1) then passes exactly through the needle guidance (23).

With regard to the production and detection of the second laser beam projection, only the change in position (32, 33) of the first projection surface (2) is decisive. More precisely, only the position (32, 33) of the first projection surface (2) relative to the guide laser beam (1) is decisive. It follows that the required change in position (32, 33) may be achieved in different ways. For example, the alignment facility as a whole may be displaced by moving the robotic arm (21). For example, the alignment facility may be displaced relative to the robotic arm (21). And, for example, the first projection surface (2) may be displaced relative to the camera (7). In the latter variant, changed mapping ratios may result when the first projection surface is detected by the camera (7), and this may have to be taken into account when determining the position of the laser beam projection within the projection surface (2).

FIG. 2 is a schematic illustration of an alignment facility according to an embodiment. With the exception of the linear adjuster (6), the alignment unit includes the same components as described in the preceding FIG. 1. In this respect, reference is made to the above explanations.

A linear adjuster may for example be an electromechanical device that produces a linear movement in order to adjust the position of an instrument linearly along a spatial axis. A linear adjuster may be electrically or manually operated. It may ensure high-precision spatial alignment. Alternative terms may be linear drive or linear motion unit. An example of a linear adjuster is a motorized displacement mechanism that provides for exact placement of a biopsy needle along a spatial axis.

The alignment facility includes a linear adjuster (6) by which the camera (7) and the first projection surface (2) may be displaced such that the position (32, 33) of the first projection surface (2) changes. The position (32, 33) of the first projection surface (2) may be changed by the linear adjuster (6) such that it is displaced by the distance D relative to its initial position (32, 33). In this figure, the first projection surface (2) is in the first projection surface position (33). The linear adjuster (6) may be used to displace it by the distance D to the second projection surface position (32). The linear adjuster (6) serves to increase the precision of the linear movement. The movement may, for example, be executed as a vertical movement. For example in the case of movements of a robotic arm (21), there is a risk that, due to tolerances in the robotic arm (21), these movements will not be exactly vertical and will not be exactly by the distance D, resulting in slightly deviating movements. Such inaccuracies caused by the manipulator may be eliminated by a linear adjuster (6) that enables high-precision linear movements.

In the first projection surface position (33), the guide laser beam (1) produces a first laser beam projection in a first position (4). As explained above, the first laser beam projection may be detected by the camera (7) so that its position (4) may be ascertained using the camera (7).

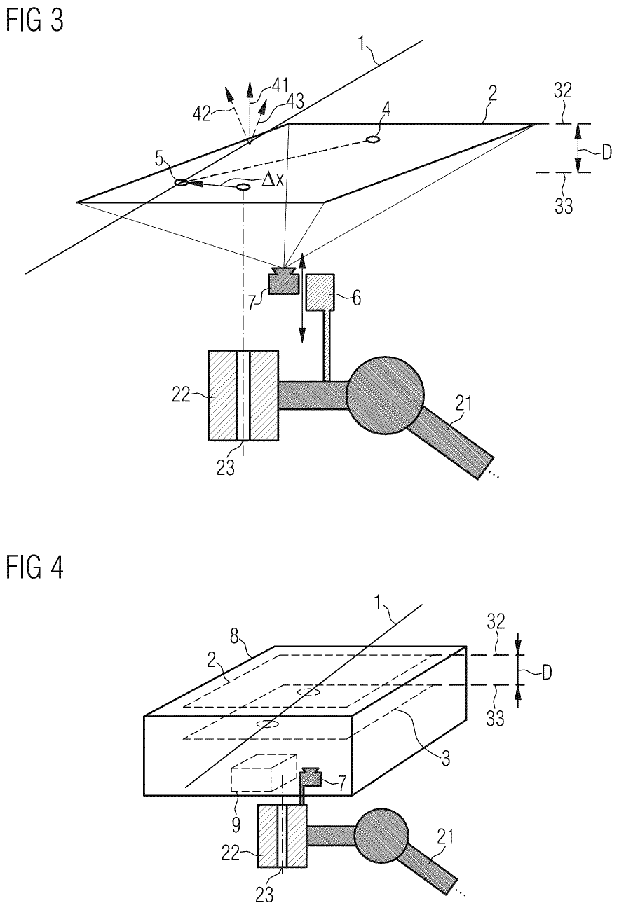

FIG. 3 is a schematic illustration of the alignment facility explained in FIG. 2 according to an embodiment with a changed projection surface position (32, 33). In contrast to the above-described illustration, the first projection surface (2) is now in the second projection surface position (32) to which it was displaced by the linear adjuster (6).

In the second projection surface position (32), the guide laser beam (1) produces a second laser beam projection in a second position (5). As explained above, the second laser beam projection is detected by the camera (7) so that its position (5) may be ascertained using the camera (7). As explained above, the angle α at which the guide laser beam (1) strikes the first projection surface (2) and the spatial alignment of the angle α may be calculated based on the two positions (4, 5) of the laser beam projections and the distance D. Based on the angle information ascertained in this way, the robotic arm (21) and thus the needle guide sleeve (22) may be aligned with the guide laser beam (1).

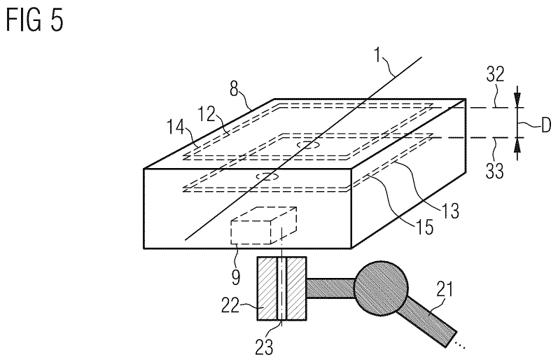

FIG. 4 is a schematic illustration of an alignment apparatus (8) according to an embodiment. The alignment apparatus (8) includes an optical detection facility configured as a camera (7). The camera (7) is aligned so that it may detect first and second laser beam projections produced in a first and second projection surface position (32, 33). The alignment apparatus further includes a computing facility (9) for receiving and processing the data detected by the camera (7). The computing facility (9) is configured to ascertain the positions (4, 5) of laser beam projections detected by the camera (7). It is further configured to ascertain the angle α and its alignment in relation to the guide laser beam (1) based on these positions (4, 5) and the distance D between the projection surface positions (32, 33).

Laser beam projections in the first and second projection surface position (32, 33) are produced through a first projection surface (2) in the second projection surface position (32) and through a further, second projection surface (3) in the first projection surface position (3). The first projection surface (2) is transparent so that a guide laser beam (1) may pass through it. When it strikes and passes through the first projection surface (2), the guide laser beam (1) produces a first laser beam projection. After passing through the first projection surface (2), the guide laser beam (1) strikes the second projection surface (3), where it produces a second laser beam projection.

In the arrangement selected for the illustration, the camera (7) is located below both projection surfaces (2, 3). Like the first projection surface (2), the second projection surface (3) is transparent so that the camera (7) may detect the second laser beam projection from below, i.e., from the side facing away from the guide laser beam. Because the second projection surface (3) is transparent, the camera (7) may also detect the first laser beam projection through the second projection surface (3). As an alternative to the arrangement selected for the illustration, the camera (7) may also be arranged between the two projection surfaces (2, 3). In this case, the second projection surface (3) may not necessarily have to be transparent. As a further alternative, the camera (7) may also be arranged above the two projection surfaces (2, 3). In this case, the second projection surface (3) may likewise not necessarily have to be transparent.

Instead of an alignment facility with a first and second projection surface (2, 3), the alignment apparatus (8) may alternatively also include only a first projection surface (2). In this embodiment, the first projection surface (2) may have to be moved linearly between the detection of the first laser beam projection and the second laser beam projection by the distance D. As explained above, this may be achieved either by the robotic arm (21) displacing the alignment apparatus (8) or by the alignment apparatus (8) being displaced relative to the robotic arm (21), for example by a linear adjuster, or by the first projection surface (2) being displaced within the alignment apparatus (8), for example by a linear adjuster.

In an embodiment including only a first projection surface (2), the computing facility (9) first ascertains the first laser beam projection or its position (4) in the first projection surface (2). It then initiates the linear displacement of the first projection surface (2) by issuing a command to this effect. After the displacement, it ascertains the second laser beam projection or its position (5) in the first projection surface (2).

When the computing facility (9) has ascertained the positions (4, 5) of the first and second laser beam projection, it calculates the angle α and its alignment. The information calculated by the computing facility (9) regarding the angle α and its alignment is then provided as alignment information by the alignment apparatus (8). This may be transmitted to a control facility via a suitable data connection. The data connection may be either wireless or wired. Based on the alignment information received, the control facility may initiate a change in the alignment of the robotic arm (21).

The alignment apparatus (8) may be attached detachably to the robotic arm (21). In this case, the alignment apparatus (8) may be detached from the robotic arm (21) after the robotic arm (21) has been aligned. Instead of the alignment apparatus (8), an instrument may then be attached to the robotic arm (21), for example a needle guide sleeve (22). The alignment apparatus (8) may be attached to the robotic arm (21) by a flange connection that matches that of the instrument. In this way, the alignment apparatus (8) may be used as a reusable separate apparatus for aligning different manipulators and instruments.

FIG. 5 is a schematic illustration of a further embodiment according to an embodiment. It largely corresponds to the embodiment illustrated and explained above in FIG. 4. In this respect, reference is made to the preceding explanations.

In contrast to the above-explained embodiments, the embodiment illustrated in FIG. 5 does not have a camera as an optical detection facility. Instead of using a camera, the laser beam projection is optically detected by a sensitive layer (14, 15) on the respective projection surface (12, 13). The respective layer (14, 15) is sensitive to the wavelength range of the guide laser beam (1). The sensitive layer (14, 15) effectively turns the respective projection surface (12, 13) into an image sensor for light from the guide laser beam (1). The sensitive layer (14, 15) may be used to optically detect the position on the respective projection surface (12, 13) at which the guide laser beam (1) strikes the layer (14, 15) and thus the projection surface (12, 13) and produces a laser beam projection.

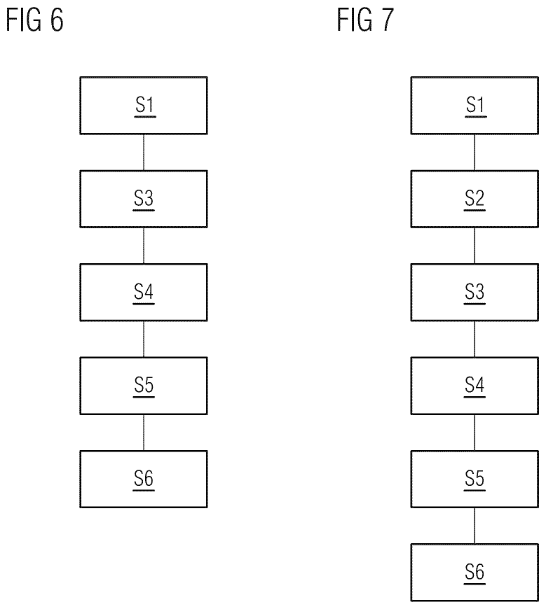

FIG. 6 is a schematic illustration of the method. With regard to the terms used, reference is made to the descriptions of the preceding figures. The same terms are used with the same meaning.

In step S1), the optical detection facility detects a first laser beam projection.

In step S3), the optical detection facility detects a second laser beam projection. Herein, the second laser beam projection is produced in a different projection surface position (32, 33) than the first laser beam projection. Herein, the projection surface positions (32, 33) differ by the distance D.

In step S4), the detected laser beam is used to ascertain its respective position (4, 5) within the respective projection surface (2, 3, 12, 13) and in the respective projection surface position (32, 33).

In step S5), the ascertained positions (4, 5) of the first and second laser beam projection and the distance D between the respective projection surfaces (2, 3, 12, 13) or projection surface positions (32, 33) are used to calculate the angle α and its alignment in relation to the projection surfaces (2, 3, 12, 13) or to the alignment facility.

In addition to calculating the angle α and its spatial alignment, the ascertained positions (4, 5) of the first and second laser beam projection also provide for the calculation of the correction distance Δx in order to be able to align not only the spatial orientation, but also the spatial position of the alignment facility or the robotic arm (21).

In step S6), the information regarding the angle α and its alignment are provided as alignment information.

FIG. 7 is a schematic illustration of the method. The method supplements the above-described method. The same method steps have the same names and are used interchangeably.

In step S2), in addition to the above-described method, after the detection of a first laser beam projection and before the detection of a second laser beam projection, the alignment facility is moved linearly. It is essential that the projection surface (2, 3, 12, 13) is moved from the first projection surface position (32, 33) to the second projection surface position (32, 33) or vice versa. Step S2) is particularly necessary if the alignment facility only includes a first projection surface (2) and not a second projection surface (3).

It is to be understood that the elements and features recited in the appended claims may be combined in different ways to produce new claims that likewise fall within the scope of the present disclosure. Thus, whereas the dependent claims appended below depend from only a single independent or dependent claim, it is to be understood that the dependent claims may, alternatively, be made to depend in the alternative from any preceding or following claim, whether independent or dependent, and that such new combinations are to be understood as forming a part of the present specification.

While the present disclosure has been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

1. An alignment facility for aligning a manipulator with a guide light beam, the alignment facility comprising:
a first projection surface configured to produce a first projection of a guide light beam incident on the first projection surface; and
an optical detection facility configured to detect a first projection produced on the first projection surface;
wherein the first projection surface is arranged on the manipulator in a fixed relative spatial position with respect to the manipulator in relation to at least two spatial axes, and wherein the optical detection facility is arranged on the manipulator in a fixed relative spatial position with respect to the first projection surface.

2. The alignment facility of claim 1, wherein the alignment facility is configured to be arranged detachably on the manipulator.

3. The alignment facility of claim 1, wherein the alignment facility is configured to be arranged on the manipulator in a fixed relative spatial position with respect to the manipulator.

4. The alignment facility of claim 1, wherein the alignment facility is configured to be arranged on the manipulator in a linearly displaceable relative spatial position with respect to the manipulator.

5. The alignment facility of claim 1, wherein the first projection surface is transparent.

6. The alignment facility of claim 5, further comprising:
a second projection surface, wherein the first projection surface and the second projection surface are arranged one above the other at a distance from one another so that a guide light beam strikes the first projection surface and then the second projection surface;
wherein both the first projection surface and the second projection surface are configured to each produce a projection of a guide light beam incident on the respective projection surface;
wherein both the first projection surface and the second projection surface are configured to be arranged on the manipulator in a fixed relative spatial position with respect to the manipulator;
wherein the optical detection facility is configured to detect a projection produced on a respective projection surface.

7. The alignment facility of claim 6, wherein the distance between the first projection surface and the second projection surface is 10 to 50 mm.

8. The alignment facility of claim 1, wherein the optical detection facility is configured as a layer that is sensitive to the wavelength of the guide light beam, and wherein the first projection surface comprises the layer or is connected thereto.

9. The alignment facility of claim 1, further comprising:
a computing facility configured to receive and process projection information detected by the optical detection facility.

10. The alignment facility of claim 9, wherein the computing facility is configured to ascertain alignment information using at least two items of projection information received from the optical detection facility.

11. The alignment facility of claim 9, wherein the computing facility is configured to initiate a linear spatial displacement of the alignment facility.

12. A method for aligning a manipulator with a guide light beam using an alignment facility, the method comprising:
detecting, by an optical detection facility, a first projection of a guide light beam incident on a first projection surface produced on a first projection surface in a first projection surface position;
detecting, by the optical detection facility, of a second projection of the guide light beam produced on the first projection surface or a second projection surface in a second projection surface position, wherein the first projection surface position and the second projection surface position are located one above the other at a distance from one another;
ascertaining first position information and second position information describing a respective position of the first projection and second projection within the respective projection surface;
ascertaining an angle between the guide light beam and the projection surfaces based on the first position information and the second position information by a computing facility; and
providing the angle.

13. The method of claim 12, further comprising:
after the detection of the first projection and before the detection of the second projection of the guide light beam, linearly moving the alignment facility with respect to the manipulator in such a way that the first projection surface is moved from the one projection surface position to the other projection surface position in each case.

14. The method of claim 12, further comprising:
after the detection of the first projection and before the detection of the second projection of the guide light beam, linearly moving the manipulator in such a way that the first projection surface is moved from one projection surface position to the other projection surface position in each case.

15. A non-transitory computer implemented storage medium, including machine-readable instructions stored therein for aligning a manipulator with a guide light beam using an alignment facility, the machine-readable instructions when executed by at least one processor, cause the processor to:
detect a first projection of a guide light beam incident on a first projection surface produced on a first projection surface in a first projection surface position;
detect of a second projection of the guide light beam produced on the first or second projection surface in a second projection surface position, wherein the first projection surface position and the second projection surface positions are located one above the other at a distance from one another;
ascertain first position information and second position information describing a respective position of the first projection and second projection within the respective projection surface;
ascertain an angle between the guide light beam and the projection surfaces based on the first position information and the second position information by a computing facility; and
provide the angle.
